(19) United States
(12) Patent Application Publication
(10) Pub. No.: US 2009/0275143 A1
(43) Pub. Date: Nov. 5, 2009

(54) NANOSTRUCTURE ARRAY AND METHODS OF USE FOR EXPLOSIVE DETECTION

(75) Inventors: Manoranjan Misra, Reno, NV (US); Susanta K. Mohapatra, Reno, NV (US); Subarna Bannerjee, Reno, NV (US); Indu B. Mishra, Scottsdale, AZ (US)

Correspondence Address: UNR/DRI Technology Transfer Office, UNR-DRI Technology Transfer Office, Mail stop 321, 1664 N. Virginia St, Reno, NV, 89557, US

(73) Assignee: The Board of Regents of the Nevada System of Highe Education, on Behalf of the University of Nevada,

(21) Appl. No.: 12/399,838

(22) Filed: Mar. 6, 2009

Related U.S. Application Data
(60) Provisional application No. 61/034,705, filed on Mar. 7, 2008.

(51) Int. Cl. G01N 33/22; G01N 27/00

(52) U.S. Cl. 436/130; 436/150; 422/82.01; 977/742

(57) ABSTRACT

In various embodiments, the present disclosure provides a method of detecting triacetone triperoxide. In a particular implementation, the method includes providing an array of titanium nanostructures that include a sensitizing agent. The array is contacted with a fluid sample, such as vapor sample. The resistance of the array is measured. The measured resistance of the array can be used to determine whether the sample includes triacetone triperoxide. The nanostructures are, in some cases, titania nanotubes. The sensitizing agent is, in a specific example, zinc. The present disclosure also provides a triacetone triperoxide sensor that includes an array of nanostructures, at least a portion of which are sensitized with a sensitizer, such as one or more of Li⁺, Cu²⁺, In³⁺, Sb³⁺, Sc³⁺, NH₄⁺, Na⁺, Zn²⁺, Cd²⁺, and Ti⁴⁺. A plurality of electrical contacts are coupled to the array.

### CROSS REFERENCE TO RELATED APPLICATION

This application claims the benefit of, and incorporates by reference, U.S. Provisional Patent Application No. 61/034,705, filed Mar. 7, 2008.

### TECHNICAL FIELD

The present disclosure relates to nanostructure arrays and their use. In particular examples, the present disclosure concerns a method for detecting explosives, such as triacetone triperoxide, using an array of sensitized titanium nanotubes.

### BACKGROUND

Conventional ammonium nitrate-fuel oil (ANFO) explosives are used extensively for blasting operations. These explosives have the potential to be used by terrorists. Over the past several years, researchers have developed a variety of detectors for nitrate based explosives. Recently, however, the most actively used homemade explosive is triacetone triperoxide (TATP), particularly in Israel, U.K. and the U.S.

TATP can easily be made, is highly potent, and little research has focused on its detection. The explosion of TATP involves entropy burst, which results from the formation of one ozone and three acetone molecules from every molecule of TATP in the solid state. Just a few hundred grams of the material can produce hundreds of liters of gas in a fraction of a second (a 10 g sample produced 250 cm³ expansions in the Trauzl test).

Typical methods of TATP detection are not rapid or selective enough for field use. For example, Fourier transform infrared spectroscopy (FTIR) and gas chromatography-mass spectroscopy (GC-MS) have been used for TATP detection, but are more applicable to a forensic lab. TATP has also been identified by desorption electrospray ionization (DESI) mass spectrometry. However, this method typically requires significant time for ionization and desorption. DESI also typically uses sophisticated corona or glow discharge and chemical ionization mass spectroscopy, which can make it awkward for field use.

### SUMMARY

In one aspect, the present disclosure provides a method of detecting triacetone triperoxide. According to the method, an array of titanium nanostructures having a sensitizing agent is provided. A gaseous sample is passed over the array and the resistance of the array is measured. The measured resistance is compared with a threshold value. Based on this measurement, a determination is made whether the sample includes triacetone triperoxide.

In one specific example, the nanostructures are titania nanotubes. In further examples, the sensitizing agent is selected from Li⁺, Cu²⁺, In³⁺, Sb³⁺, Sc³⁺, NH⁺, Na⁺, Zn²⁺, Cd²⁺, and Ti⁴⁺, such as Zn²⁺. The sensitizing agent may be included in the nanostructures in a number of ways, such as being coated on the surface of the nanostructures or being incorporated into the nanostructures. In another specific method, the method is carried out in an inert atmosphere. The method may include additional steps, such as irradiating the array with ultraviolet radiation, such as to help regenerate the array.

In another aspect, the present disclosure provides a triacetone triperoxide sensor having an array of nanostructures. The array includes a sensitizer selected from Li⁺, Cu²⁺, In³⁺, Sb³⁺, Sc³⁺, NH⁺, Na⁺, Zn²⁺, Cd²⁺, and Ti⁴⁺. In a particular example, the sensitizer is Zn²⁺. A plurality of electrical contacts are in electrical communication with the array.

In various examples, the sensitizer is coated on a surface of the nanostructures, such as a luminal surface, or incorporated into the nanostructures. In some examples, the nanostructures are hollow. The sensor may include additional components, such as an ultraviolet lamp.

In yet another aspect, the present disclosure provides an apparatus useful for detecting triacetone triperoxide. The apparatus includes a nanostructure array sensor as described above. The array is disposed in a housing having an inlet. A controller, such as a potentiostat, is in communication with the sensor. The apparatus may include a computer, such as to process or display information, or to control system components, such as the potentiostat. The apparatus may include additional components, such as an ultraviolet lamp, a purge gas source, or a pump. The housing includes an outlet, in some examples.

An embodiment of the present disclosure provides a method of detecting a chemical substance. According to the method, an array of nanostructures is provided, the nanostructures having a peroxide-complexing sensitizing agent. A gaseous sample is passed over the array and the resistance of the array is measured. The resistance is compared with a threshold value and a determination is made whether the sample contains triacetone triperoxide.

In some examples, the nanostructures are TiO₂ nanostructures, such as TiO₂ nanotubes. In further examples, the sensitizing agent is selected from Li⁺, Cu²⁺, In³⁺, Sb³⁺, Sc³⁺, NH⁺, Na⁺, Zn²⁺, Cd²⁺, and Ti⁴⁺. In a specific example, the sensitizing agent is Zn²⁺.

In another implementation of the method, the array is obtained by treating an unsensitized array of nanostructures to exchange at least a portion of exchangeable sites on the unsensitized array with the sensitizing agent.

There are additional features and advantages of the subject matter described herein. They will become apparent as this specification proceeds.

In this regard, it is to be understood that this is a brief summary of varying aspects of the subject matter described herein. The various features described in this section and below for various embodiments may be used in combination or separately. Any particular embodiment need not provide all features noted above, nor solve all problems or address all issues in the prior art noted above.

### BRIEF DESCRIPTION OF THE DRAWINGS

Various embodiments are shown and described in connection with the following drawings in which:

FIG. 1 is a schematic diagram illustrating a metal ion sensitized TiO₂ nanotube sensor interacting with TATP.

FIG. 2 is a schematic diagram illustrating one disclosed implementation of a TATP detection apparatus or system.

FIG. 3 presents a schematic diagram of an apparatus for sonoelectrochemical anodization.

FIG. 4 presents a schematic diagram of an apparatus using a TiO₂ nanotube sensor to detect TATP vapor.

FIG. 5A is a circuit diagram of a detector useable in the apparatus of FIG. 4.

FIG. 5B is a circuit diagram of a UV regeneration component useable in the apparatus of FIG. 4.

FIG. 6 is a FESEM image of a TiO₂ nanotube array prepared according to a method of the present disclosure.

FIG. 7 is a graph of current versus potential for several nanotube arrays formed using different methods.

FIG. 8 is a graph of current versus time for a Zn²⁺ sensitized TiO₂ nanoarray in an argon atmosphere.

FIG. 9 is a graph of current versus time for a Zn²⁺ sensitized TiO₂ nanoarray in an argon atmosphere and in the presence of TATP vapor.

FIG. 10 is a FESEM image of a TiO₂ nanotube array prepared according to a method of the present disclosure.

FIG. 11 is an O1s XPS spectrum of a TiO₂ nanotube array prepared according to a method of the present disclosure.

FIG. 12 is a graph of current versus time for a Zn²⁺ sensitized TiO₂ nanoarray in an argon atmosphere without TATP.

FIG. 13 is a graph of current versus time for a Zn⁺² sensitized TiO₂ nanoarray as the nanoarray is exposed to an argon atmosphere without TATP, TATP is added to the nanoarray, and the nanoarray is again exposed only to an argon atmosphere.

FIG. 14 is a graph of current versus time for a Zn⁺² sensitized TiO₂ nanoarray as the nanoarray is exposed to an ambient atmosphere without TATP, TATP is added to the nanoarray, and the nanoarray is again exposed only to an ambient atmosphere.

### DETAILED DESCRIPTION

Unless otherwise explained, all technical and scientific terms used herein have the same meaning as commonly understood by one of ordinary skill in the art to which this disclosure belongs. In case of conflict, the present specification, including explanations of terms, will control. The singular terms “a,” “an,” and “the” include plural referents unless context clearly indicates otherwise. Similarly, the word “or” is intended to include “and” unless the context clearly indicates otherwise. The term “comprising” means “including;” hence, “comprising A or B” means including A or B, as well as A and B together. Although methods and materials similar or equivalent to those described herein can be used in the practice or testing of the present disclosure, suitable methods and materials are described herein. The disclosed materials, methods, and examples are illustrative only and not intended to be limiting.

The following definitions are provided in order to aid in understanding the discussion of certain embodiments of the present disclosure which follow.

“Brightening agents” refers to compounds that impart luster to a deposited material, and include semibrighteners. Brightening agents can also affect grain size and shape, as well as the throwing power of an electrochemical bath, such as a deposition bath. A number of brightening agents are discussed in U.S. Pat. No. 4,384,930 to Eckles, which is expressly incorporated by reference herein to the extent not inconsistent with the present disclosure. In some embodiments, the brightening agent is a surfactant, such as a non-ionic surfactant. In further embodiments, the brightener or semibrightener is selected from epihalohydrin; 2,4-diamino-6-(2′-methylimidazol-1′-yl)ethyl-1,3,5-triazine; 2,4-diamino-6-(2′-ethyl-4-methylimidazol-1′-yl)ethyl-1,3,5-triazine; 2,4-diamino-6-(2′-undecylimidazol-1′-yl)ethyl-1,3,5-triazine; aldehydes, such as cinnamaldehyde, acetaldehyde, salicylaldehyde, crotonaldehyde, benzaldehyde, o-chlorobenzaldehyde, p-chlorobenzaldehyde, 2,4-dichlorobenzaldehyde, 2,4,6-trichlorobenzaldehyde, m-chlorobenzaldehyde, p-nitrobenzaldehyde, p-hydroxybenzaldehyde, glutaraldehyde, paraldehyde, 2-thiophenaldehyde, 3-thiophenaldehyde, o-anisaldehyde, m-anisaldehyde, p-anisaldehyde, salicylaldehyde allyl ether, vanillin and like aldehydes, 1-naphthaldehyde, 2-naphthaldehyde, 2-hydroxy-1-naphthaldehyde, and 3-acenaphthaldehyde; 2-methylbenzothiazole; 2-aminobenzothiazole; 2-mercaptobenzothiazole and like benzothiazoles; triazine; imidazole; indole; quinoline; 2-vinylpyridine; aniline; benzylideneacetone; pyridydeneacetone; furfurylideneacetone; furfural; acrolein; benzothiazole; phenanthroline; neocuproine; picolinic acid; thioureas; N-(3-hydroxybutylidene)-p-sulfanilic acid; N-butylidenesulfanilic acid; N-cinnamoylidenesulfanilic acid; phenyl salicylate; polyamines, such as ethylenediamine, ethylenediaminetetraacetic acid (EDTA), hydroxyethylethylenediaminetriacetic acid (HEDTA), triethylenetetraminehexaacetic acid (TTHA), triethylenetetraminehexaacetic acid (TTHA), ethylenedioxybis(ethylamine)-N,N,N′N′-tetraacetic acid, diethylene-triaminepentaacetic acid (DTPA), ethylenetriamine, N-hydroxyethylenediamine (HEEDA), 1,2-diaminocyclohexane-N,N,N′,N′-tetraacetic acid, 1,3-diaminohydroxypropaney-N,N,N′,N′-tetraacetic acid; diethylenetriamine-N,N,N′,N′,N′-pentaacetic acid; and salts, other derivatives, and combinations thereof.

“Carrier” refers to a substance added to a plating bath to help provide uniform deposition. For example, carriers can coat the surface of a deposit, thus limiting access to other substances. Carriers can also help fill holes or gaps in the surface of a deposit. Suitable carriers include polyethers and polyoxyethers. For example, the carrier may be a polyalkylene glycol, such as polyethylene glycol.

“Complexing agent” refers to compounds that can be used to increase the solubility of metals in the bath solution, or otherwise adjust the availability of metal ions for deposition. Complexing agents can also be used to increase the Faradic efficiency (and therefore the throwing power) of solutions. Complexing agents can also be added to a solution of a sensitizing agent in order to increase the solubility of the agent or to alter the properties of the solution, such as the ease with which the sensitizing agent may exchange with exchangeable sites on a nanostructure. The complexing agent can also serve other functions, such as acting as a brightening agent, in which can the same species can serve multiple functions.

Common complexing agents include anions of metal salts, such as halides, sulfates, sulfites, thiosulfates, nitrates, nitrites, cyanides, or thiocyanates. In some examples, the complexing agent is selected from oxycarboxylic acids, monocarboxylic acids, and polycarboxylic acids, and salts, other derivatives, and combinations thereof. Suitable examples of such acids include gluconic acid, glucoheptonic acid, oxalic acid, citric acid, tartaric acid, lactic acid, malic acid, malonic acid, acetic acid, succinic acid, gluconolactone acid, diglycolic acid, ascorbic acid, propionic acid, glucoheptlactone, formic acid, butyric acid, diglycolic acid, and salts, other derivatives, and combinations thereof. Suitable complexing agents further include disulfides, such as dithiodianiline and dithiodipyridine; thiocarboxylic acids, such as acetylcysteine and mercaptosuccinic acid; amino acids and thioamino acids, such as cysteine and methionine; thiourea and thiourea derivatives, such as trimethyl thiourea and allyl thiourea; sulfides, such as dimethyl sulfoxide (DMSO); and salts, other derivatives, and combinations thereof.

Aldehyde compounds may also be used as complexing agents. Suitable examples include 2-thiophenaldehyde; 3-thiophenaldehyde; 1-naphthaldehyde; 2-naphthaldehyde; acetaldehyde; salicylaldehyde; o-anisaldehyde; m-anisaldehyde; p-anisaldehyde; salicylaldehyde allyl ether; o-chlorobenzaldehyde; m-chlorobenzaldehyde; p-chlorobenzaldehyde; 2,4-dichlorobenzaldehyde; and derivatives and combinations thereof.

Complexing agents can also include polyamines, such as ethylenediamine; ethylenediaminetetraacetic acid (EDTA); hydroxyethylethylenediaminetriacetic acid (HEDTA); triethylenetetraminehexaacetic acid (TTHA); ethylenedioxybis(ethylamine)-N,N,N′N′-tetraacetic acid; diethylene-triaminepentaacetic acid (DTPA); ethylenetriamine; N-hydroxyethylenediamine (HEEDA); 1,2-diaminocyclohexane-N,N,N′,N′-tetraacetic acid; 1,3-diaminohydroxypropaney-N,N,N′,N′-tetraacetic acid; diethylenetriamine-N,N′,N′,N″,N″-pentaacetic acid; and salts, other derivatives, and combinations thereof.

In further embodiments, the complexing agent is a substance selected from glycines; nitrilotrimethyl phosphonic acid; 1-hydroxyethane-1,1-diphosphoric acids; N,N-bis(2-hydroxyethyl)glycine; iminodiacetic acid; nitrilotriacetic acid; nitrilotripropionic acid; nitrilotriacetic acid (NTA); iminodiacetic acid (IDA); iminodipropionic acid (IDP); diethanolamine (DEA); triethanolamine (TEA); N-methylethanolamine; and 2-aminopropanol; and salts, other derivatives, and combinations thereof.

“Grain refiner” refers to one or more substances added to a plating bath to promote a more uniform surface or a less porous surface. Suitable grain refiners in some embodiments of the present disclosure include surfactants, such as anionic and non-ionic surfactants. Suitable nonionic surfactants are available from Rohm & Haas Company, Philadelphia, Pa., under the tradenames TRITON CF-54, TRITON DF-18, TRITON D-18, TRITON D-16, and TRITON CF-76. Additional surfactants include ANTAROX F-330, available from GAF Chemicals Corp. of Wayne, N.J., and AVANEL polyether surfactants, available from PPG of Pittsburgh, Pa.

Further examples of grain refiners include 4-phenyl-3-buten-2-one; phenolphthalein; pyridine carboxylic acids, such as picolinic acid, nicotinic acid, and, isonicotinic acid; alkoxylates, such as polyethoxylated amines or sulfated alkyl ethoxylates; gelatin and gelatin derivatives; lower alkyl (C₁-C₆) alcohols; and polyalkylene glycols, such as ethylene glycol.

“Leveling agent” refers to one or more substances added to a plating bath to promote more uniform layer growth. Leveling agents thus help produce a smooth surface and can cause the thickness of a coating to vary based on the underlying surface features of the substrate, such as having thicker deposits in recesses and thinner deposits over protrusions. Suitable leveling agents in some embodiments of the present disclosure include surfactants, such as anionic and non-ionic surfactants. In some examples, leveling agents are selected from amines and amide surfactants. Suitable nonionic surfactants are available from Rohm & Haas Company, Philadelphia, Pa., under the tradenames TRITON CF-54, TRITON DF-18, TRITON D-18, TRITON D-16, and TRITON CF-76. Additional surfactants include ANTAROX F-330, available from GAF Chemicals Corp. of Wayne, N.J., and AVANEL polyether surfactants, available from PPG of Pittsburgh, Pa. Further examples of surfactants useable as leveling agents are provided in the definition of “surfactant” provided in the present disclosure.

In further examples, the leveling agent includes a polyalkylene glycol or a derivative thereof, such as polyethylene glycol; allyl alcohol; saccharin; alkylated polyalkyleneimine; ethylene thiourea; 1-(2-hydroxyethyl)-2-imidazolidinethione; imidazole; 4-mercaptopyridine; 2-mercaptothiazoline; polyvinyl carbamates; pyrrolidone; thiourea; polymeric amines; triisopropanolamine; alkylated polyalkyleneimine, such as tetrapolypropoxy-ethoxy ethylenediamine sulfosuccinic ester sodium salt; or 4-mercaptopyridine.

“Nanostructure” refers to a solid structure having a cross sectional diameter of between about 0.5 nm to about 500 nm. Nanostructures may be made from a variety of materials, such compounds of titanium, silicon, zirconium, aluminum, cerium, yttrium, neodymium, iron, antimony, silver, lithium, strontium, barium, ruthenium, tungsten, nickel, tin, zinc, tantalum, molybdenum, chromium, and mixtures thereof. Suitable compounds include transition metal chalcogenides or oxides, including mixed metal and/or mixed chalcogenide and/or mixed oxide compounds. In particular examples, the nanostructure is made from one or more of zinc oxide, titanium dioxide, gallium nitride, indium oxide, tin dioxide, magnesium oxide, tungsten trioxide, and nickel oxide.

In at least some examples, one or more materials from which the nanostructure is made are semiconductors. In some examples, the material has a band gap of at least about 2 eV, such as between about 2 eV and about 5 eV, between about 2 eV and about 4 eV, or between about 2 eV and about 3 eV, such as about 2.5 eV. In yet further examples, the material has a band gap of less than about 4 eV. In particular examples, the nanostructures have a resistivity lower than about 10⁻³ Ωm, such as less than about 10⁻⁶ Ωm or less than about 10⁻⁷ Ωm, such as between about 10⁻¹⁴ Ωm and about 10⁻¹⁰ Ωm or between about 10⁻¹² Ωm and about 10⁻⁶ Ωm. In some embodiments, the nanostructures have a resistivity of about 10⁻¹² Ωm.

The nanostructure can be formed in a variety of shapes. In one implementation, the nanostructures are wires, such as wires having at least one cross sectional dimension less than about 500 nm, such as between about 0.5 nm and about 200 nm. Nanowires can be nanorods, having a solid core, or nanotubes having a hollow core. In some implementations, the cross sectional dimension of the nanostructure is relatively constant. However, the cross sectional dimension of the nanostructure can vary in other implementations, such as rods or tubes having a taper.

“Pulse electrolysis” refers to electrochemical methods where current is applied in a time varying manner, as opposed to constant, direct current techniques. Pulse electrolysis can be used in different techniques, such as anodization or electrodeposition.

Pulse electrolysis can result in a more uniform or homogenous surface. When used for electrodeposition, pulse electrolysis can result in fine grain deposition. Compared with direct current techniques, pulse current techniques can allow a higher instantaneous current density to be delivered to the anode. These techniques can be applied in both acidic and basic bath solutions. Acidic solutions tend to be more efficient, but can result in more than one phase being formed. In some embodiments of the present disclosure, it can be advantageous to have a less homogenous coating, or one having more than one phase, as these qualities can produce more exchangeable sites which can then be sensitized using a desired agent, as described elsewhere in this disclosure.

In particular examples, pulse electrolysis is carried out at a temperature of between about 15° C. and about 120° C., such as between about 20° C. and about 80° C., such as at 25° C. The cathodic density is typically between about 0.1 A cm⁻² and about 20 A cm⁻², such between about 3 A cm⁻² and about 10 A cm⁻² or about 6 A cm⁻². Current on time is typically between about 0.05 ms and about 5 s, such as between about 0.1 ms and about 10 ms. Current off time is typically between about 0.05 ms and about 400 s, such as between about 0.25 ms and about 9 ms. An anodic pulse is applied, in some examples, during all or part of the current off time, such as for a period of between about 0.05 ms and 50 ms, such as between about 0.25 ms and about 15 ms. In further examples, the current off time is used as a rest period and no current is applied during this time.

Cathodic current density is typically between about 0.005 A cm⁻² and about 200 A cm⁻², such as between about 1 A cm⁻² and about 100 A cm⁻² or between about 1 A cm⁻² and about 10 A cm⁻². Anodic current density is typically between about 0.05 A cm⁻² and about 1 cm⁻², such as between about 0.1 A cm⁻² and about 0.5 A cm⁻². In at least some examples, finer grain deposits can be formed by increasing the electrolytic parameters, such as increasing the cathodic current density, the anodic current density, the cathodic on time, and the cathodic off time.

An increase in cathodic density can result in a smaller grain size and a higher nucleation rate. An increase in cathodic on time can lower surface roughness, as it can decrease grain size and result in more spherical grains. Although increasing current time-off can result in finer grain sizes, current times that are too long, such as greater than about 5 ms, can result in local corrosion, resulting in surface flaws. However, as noted above, in some aspects of the present disclosure, the presence of surface flaws can be beneficial in creating more exchangeable sites for a sensitizing agent. Increasing anodic (reverse) peak density also results in finer grain size and more spherical grains. Increasing the anodic current density above about 0.2 A cm⁻² can result in surface defects, such as by ionization of surface components. The potential needed to reduce (or oxidize) a particular metal in the electroplating method can be determined by standard means, including determination of the overpotential for a particular cell needed to deposit or anodize a particular substance.

“Sensitizing agent” refers to a substance added to a nanostructure to alter a nanostructure's interaction with one or more substances. For example, a sensitizing agent may cause the nanostructure, including a portion thereof, to interact more strongly with an analyte. In some implementations, the sensitizing agent is added to a surface of the nanostructure. In further implementations, the sensitizing agent is incorporated into the nanostructure, or a portion thereof. Specific examples of suitable sensitizing agents, such as to enhance detection of peroxides, include metal ions or other inorganic ions, such as Li⁺, Cu²⁺, In³⁺, Sb³⁺, Sc³⁺, NH₄⁺, Na⁺, Zn²⁺, Cd²⁺, Ti⁴⁺, or mixtures thereof.

“Stress reliever” refers to one or more substances added to a deposition bath to reduce internal stresses in a deposition layer, which can improve the quality of the resulting deposit, such as by producing coatings free or substantially free of cracks, or having fewer cracks than deposits produced in the absence of such substances. Suitable stress relievers in some embodiments of the present disclosure include surfactants, such as cationic, anionic, and non-ionic surfactants.

“Substrate” refers to a surface onto which nanostructures are attached or on which nanostructures are formed. Suitable substrates include generally inert materials, which are typically also insulating. The substrate is typically selected to be stable during the processes by which the nanostructures are placed on the substrate. For example, in some methods, the substrate is capable of withstanding relatively high temperatures, such as at least about 500° C. Examples of substrate materials include ceramics, glasses, such as silica or soda-lime glass, quartz, alumina, silica, and insulating polymers.

Prior to use, the substrate may be subjected to one or more pretreatment steps, such as cleaning steps. Cleaning steps can include treating the substrate with a solvent, such as an organic solvent, to remove impurities present on the surface of the substrate. In a particular example, the solvent is acetone. Ultrasonication may also be used to clean the surface of the substrate.

The dimensions of the substrate can be tailored to a particular application, such as the nanostructure composition, size, desired detection limit, and other components of an apparatus with which the nanostructure array will be used. In particular examples, the substrate has a thickness of between about 0.25 mm and about 2 mm, such as between about 0.5 mm and about 1 mm.

Additional materials can be placed on the substrate, such as to facilitate handling of the structure or to aid in subsequent processing steps. For example, in some methods, a layer of aluminum is deposited on the substrate prior to deposition of the material from which the nanostructures will be formed.

“Surfactant” refers to a substance having both hydrophilic and hydrophobic properties. In the present disclosure, surfactants can be added to deposition baths in order to alter the properties of a deposit or to facilitate the deposition process. For example, surfactants can promote wetting of the deposition surface. Surfactants can also act as leveling agents, stress relievers, or grain refiners. The surfactants are, in various examples for particular uses, anionic surfactants, cationic surfactants, non-ionic surfactants, or mixtures thereof. Specific examples of each class of surfactants are provided below.

“Anionic surfactants” can be used to promote nucleation and reduce internal stresses in deposited material. Anionic surfactants can also be used as leveling agents, stress relievers, and grain refiners. Suitable anionic surfactants include C₈-C₁₈ alkyl sulfates; C₈-C₁₈ fatty acid salts; C₈-C₁₈ alkyl ether sulfates having one or two moles of ethoxylation; C₈-C₁₈ alkamine oxides; C₈-C₁₈ alkyl sarcosinates; C₈-C₁₈ sulfoacetates; C₈-C₁₈ sulfo succinates; C₈-C₁₈ alkyl diphenyl oxide disulfonates; C₈-C₁₈ alkyl carboxylates; C₈-C₁₈ alpha-olefin sulfonates; methyl ester sulfonates; and salts, other derivatives, and combinations thereof. The C₈-C₁₈ alkyl group can be straight chain (e.g., lauryl) or branched (e.g., 2-ethylhexyl). The cation of the anionic surfactant can be an alkali metal (such as sodium or potassium); ammonium; C₁-C₄ alkylammonium (mono-, di-, tri); or C₁-C₃ alkanolammonium (mono-, di-, tri-). Lithium and alkaline earth cations (such as magnesium or calcium) can also be used.

In further embodiments, the anionic surfactant is selected from sulfates and sulfonates; alkyl(or formalin condensate)-β-naphthalenesulfonic acids (or their salts); alkyl sulfonates; α-olefin sulfonates; alkylbenzenesulfonates; alkyl (or alkoxy) naphthalenesulfonates; (mono, di, tri) alkylnaphthalenesulfonate; alkyldiphenyl ether disulfonates; alkyl ether sulfonates; alkylsulfuric esters; polyoxyethylene alkyl ether sulfuric esters; polyoxyethylene alkyl phenol ether sulfuric esters; alkyl sulfoacetates; acyl methyl taurines; alkyl fatty acid glycerin sulfuric esters; hardened coconut oil fatty acid glyceryl sulfates; fatty acid amide polyoxyethylene sulfates; isethionates; alkyl sulfocarboxylic esters; alkyl sulfosuccinates; dialkyl sulfosuccinates; alkyl polyoxyethylene sulfosuccinates; alkyl sulfates; alkyl ether sulfates; sulfate esters of an alkylphenoxy polyoxyethylene ethanol; beta-alkoxy alkane sulfonates; alkylaryl sulfonates; alkyl monoglyceride sulfates; alkyl monoglyceride sulfonates; polyoxyethylene alkyl ether sulfate; polyoxyethylene alkylphenyl ether sulfate; (poly)alkylnaphthalenesulfonate; sodium (or ammonium or TEA) sulfosuccinic monooleylamides; and salts, other derivatives, and combinations thereof.

More specifically, the anionic surfactant may be, in specific examples, sodium saccharin; sodium dodecylsulfate (SDS); sodium dodecyl benzene sulfonate; sodium polyoxyethylene (EO12) nonyl ether sulfate; sodium polyoxyethylene (EO15) dodecyl ether sulfate; sodium dibutylnaphthalenesulfonate; decyl sulfates; tridecyl sulfates; polyoxyethylene (EO15) nonylphenyl ether sulfate; lauryl sulfosuccinates; linear C₁₀ diphenyl oxide disulfonates; lauryl sulfosuccinates; lauryl ether sulfates (having 1 or 2 moles of ethylene oxide); myristyl sulfates; cetyl sulfates; sodium dodecyinaphthalene sulfate; sodium dodecylsulfate; sodium oleyl sulfate; lauryl sulfates; sodium laurylsulfate; octyl sulfates; 2-ethylhexyl sulfates; and salts, other derivatives, and combinations thereof. Additional anionic surfactants are listed in McCutcheon's Emulsifiers and Detergents, 1993 Annuals, McCutcheon Division, MC Publishing Co., Glen Rock; N.J., pp. 263-266, incorporated herein by reference. Additional anionic surfactants, and classes of anionic surfactants, are disclosed in U.S. Pat. No. 3,929,678 to Laughlin et al., incorporated herein by reference.

In yet further embodiments, the anionic surfactant is selected from higher alcohol phosphoric monoesters; fatty acid soaps; alkyl carbonates; abitic acid; alkylcarboxylate; alkylphosphate; alkyl ether carboxylates; fatty acids; sulfosuccinates; sarcosinates; lauroyl sarcosinates; octoxynol or nonoxynol phosphates; taurates; cocoates; ricinoleates; oleates; stearates; tallates; fatty taurides; polyoxyalkylene alkyl ether phosphoric acids (phosphates); polyoxyalkylene alkyl phenyl ether phosphates; polyoxyalkylene phenyl ether phosphates; polyoxyethylene alkyl ether acetates; alkanoyl sarcosines; alkanoyl sarcosinates; alkanoyl methylalanine salts; and salts, other derivatives, and combinations thereof.

Specific examples of such surfactants include, but are not limited to, lauramine oxide; cocamine oxide; decylamine oxide; myristamine oxide; and similar anionic surfactants.

The surfactant may also be one or more “cationic surfactants,” such as alkylamine oleates; alkylamine hydrochlorides; alkylbenzalkonium halides; alkylaminoethyl glycine; polyoxyethylene alkyl methyl ammonium halides; hydroxyethyl alkyl imidazoline; tetra-lower alkylammonium halides; alkylpyridinium halides, such as hexadecyl pyridinium salt and dodecyl pyridinium salt; dodecyl picolinium salt; dodecyl imidazolium salt; oleyl imidazolium salt; alkylamine acetates, such as octadecylamine acetate and dodecylamine acetate; alkyltrimethylammonium halides, such as dodecyltrimethyl ammonium salt, hexadecyltrimethyl ammonium salt, and octadecyltrimethyl ammonium salt; dialkyldimethyl ammonium halides, such as dodecyldimethylethyl ammonium salt, octadecenyldimethylethyl ammonium salt, dodecyldimethyl ammonium betaine, and octadecyldimethyl ammonium betaine; alkyldimethyl benzyl ammonium halides, such as dimethylbenzyldodecyl ammonium salt, hexadecyldimethylbenzyl ammonium salt, and octadecyldimethylbenzyl ammonium salt; trimethylbenzyl ammonium salt; triethylbenzyl ammonium salt; and salts, other derivatives, and combinations thereof.

“Non-ionic surfactants” may be added to the plating bath, such as for use as brightening agents. Suitable non-ionic surfactants include aliphatic amines; amine oxides; phosphine oxides; sulfoxides; aliphatic amine polymers; carboxylic acid esters; carboxylic amides; ethoxylated alkylphenols; ethoxylated and propoxylated fatty alcohols; polyethylene glycol ethers of methyl glucose; polyethylene glycol ethers of sorbitol; ethylene oxidepropylene oxide block copolymers; ethoxylated esters of fatty (C₈-C₁₈) acids; condensation products of ethylene oxide with long chain amines or amides; alkoxylated alkyl phenols; alkoxylated alkyl naphthols; alkoxylated aliphatic monohydric alcohols; alkoxylated polyoxypropylene glycols; alkoxylated 2,4,7,9-tetramethyl-5-decyne-4,7-diol; alkoxylated ethylene diamine; alkoxylated fatty acids; alkoxylated amides and alkoxylated esters; polyalkylene oxide block copolymers derived from propylene, butylene, or styrene; polyoxyalkylene adducts (including a block copolymer of oxyethylene and oxypropylene) such as polyalkylene glycol, polyoxyalkylene sorbitan fatty acid esters; polyoxyalkylene sorbitol fatty acid esters; polyethylene glycol fatty acid esters; polyoxyalkylene glycerin fatty acid esters; polyoxyalkylene alkylamines; polyoxyalkylene condensate adducts of ethylenediamine; polyoxyalkylene alkylene fatty acid amides; polyoxyalkylene castor (and/or hardened castor) oils; polyoxyalkylene alkyl ethers (or esters); polyoxyalkylene phenyl (or alkylphenyl)ethers; polyoxyalkylene naphthyl (or alkylnaphthyl)ethers; polyoxyalkylene styrenated phenyl ethers; polyoxyalkylene bisphenol ethers; polyoxyethylenepolyoxypropylene block polymers; polyoxyalkylene alkyl phenyl formalin condensates; glycerin (or polyglycerin) fatty acid esters; pentaerythritol fatty acid esters; sorbitan mono (sesqui, tri) fatty acid esters; higher fatty acid mono(di)ethanolamides; alkyl-alkylolamides; oxyethylene alkylamines; styrenated phenol polyethoxylates; polyoxyethylene/polyoxypropylene block copolymers; cumylphenol polyethoxylates; polyoxyalkylenes; ethoxylated nonylphenols; ethoxylated octylphenols; ethoxylated dodecylphenols; ethoxylated fatty (C₆-C₂₂) alcohols including 3 to 20 ethylene oxide moieties; and salts, other derivatives, and combinations thereof.

In specific examples, the non-ionic surfactant is selected from methyl gluceth-10, PEG-20 methyl glucose distearate; PEG-20 methyl glucose sesquistearate; C₁₁-C₁₅ pareth-20; ceteth-8; ceteth-12; dodoxynol-12; laureth-15; PEG-20 castor oil; polysorbate 20; steareth-20; polyoxyethylene-10 cetyl ether; polyoxyethylene-10 stearyl ether; polyoxyethylene-20 cetyl ether; polyoxyethylene-10 oleyl ether; polyoxyethylene-20 oleyl ether; polyoxyethylene-20 isohexadecyl ether; polyoxyethylene-23 glycerol laurate; polyoxyethylene-20 glyceryl stearate; PPG-10 methyl glucose ether; PPG-20 methyl glucose ether; polyoxyethylene-20 sorbitan monoesters; polyoxyethylene 80 castor oil; polyoxyethylene-15 tridecyl ether; polyoxyethylene-6 tridecyl ether; laureth-2; laureth-3; laureth-4; PEG-3 castor oil; PEG 600 dioleate; PEG 400 dioleate; nonylphenol polyethoxylate; octylphenol polyethoxylate; dodecyl alcohol polyethoxylate; 1 butyl-1,4 diol; nonionic phenol detergents; ethylene glycol; dimethyl isoccynate; polyvinyl alcohol; polyacrylic acid; methalose; methyl cellulose; ethyl cellulose; propyl cellulose; hydroxy ethyl cellulose; carboxy methyl cellulose; polyoxyethylene cetyl ether; polyoxyethylene lauryl ether; polyoxyethylene octyl ether; polyoxyethylene octylphenyl ether; polyoxyethylene oleyl ether; polyoxyethylene sorbitan monolaurate; polyoxyethylene stearyl ether; polyoxyethylene nonylphenyl ether; dialkylphenoxy poly(ethyleneoxy)ethanol; phosphoric acid; polyhydric alcohol; glucoside; higher alcohols; phenols; alkylphenols; naphthols; alkylnaphthols; bisphenols; styrenated phenols; fatty acids; aliphatic amines; sulfonamides; and derivatives and mixtures thereof. Suitable nonionic surfactants are available from Rohm & Haas Company, Philadelphia, Pa., under the tradenames TRITON CF-54, TRITON DF-18, TRITON D-18, TRITON D-16, and TRITON CF-76. Additional surfactants include ANTAROX F-330, available from GAF Chemicals Corp. of Wayne, N.J., and AVANEL polyether surfactants, available from PPG of Pittsburgh, Pa.

Numerous other nonionic surfactants are disclosed in McCutcheon's Detergents and Emulsifiers, 1993 Annuals, published by McCutcheon Division, MC Publishing Co., Glen Rock, N.J., pp. 1-246 and 266-272; in the CTFA International Cosmetic Ingredient Dictionary, Fourth Ed., Cosmetic, Toiletry and Fragrance Association, Washington, D.C. (1991) (hereinafter the CTFA Dictionary) at pages 1-651; and in the CTFA Handbook, at pages 86-94, each incorporated herein by reference.

Nanostructure Synthesis

Presently disclosed embodiments concern using nanostructures to detect explosives, such as triacetone triperoxide (TATP). Nanostructures may be formed by any suitable method. The nanostructures are relatively homogenously disposed on the substrate in some examples. However, in further examples, the nanostructures may be unevenly distributed on the substrate or distributed in discrete zones. For example, the array may be formed in a stepped structure, such as to aid in uniformity of the nanostructures by forming the nanostructures in discrete portions, whose properties thus may be more accurately controlled.

The nanostructure may be a nanotube. One suitable method of forming nanotubes involves anodizing a metal or metal alloy source, such as titanium foil, in a suitable electrolytic solution. Suitable titanium foils can be obtained from commercial sources or can be prepared by various methods, such as sputtering. In some examples, the metal source has a thickness suitable for to produce a desired amount of oxide, or other substance, while retaining a sufficient amount of metal to aid in handling, durability, or conduction which, in some embodiments, is at least about 300 nm, such as between about 300 nm and about 10 mm or between about 1000 nm and about 4 mm. In a specific example, the metal source has a thickness of about 2 mm.

Prior to anodization, the metal source can be cleaned, such as by washing the source in an organic solvent, such as acetone, methanol, isopropanol, or mixtures thereof (including aqueous mixtures), optionally with sonication. The metal source can be further rinsed with water, such as deionized water, and dried.

In some embodiments, the electrolytic solution includes at least one acid, such as a solution of acetic acid, chromic acid, phosphoric acid, oxalic acid, hydrofluoric acid, or mixtures thereof. In more specific examples, the acidic solution also includes a fluoride compound, such as hydrogen fluoride or alkali fluorides, such as sodium fluoride or potassium fluoride. The solution includes at least about 0.1 wt % of fluoride compounds in some examples, such as about 0.5 wt % of one or more fluoride compounds. In other implementations, a basic electrolytic solution is used, such as a solution of potassium hydroxide. The electrolyte solution can include other substances.

In various examples, the anodization potential is between about 1 V and about 50 V, such as between about 5 V and about 20 V or about 10 V. Constant anodization voltage can be used to produce nanotubes having a relatively constant diameter. Ramped or stepped voltages can be used to produce shaped nanotubes, such as tapered conical nanotubes. Pulsed electrolysis can also be used.

The temperature of the anodization process can also affect the properties of the nanostructures, such as the wall thickness of nanotubes. Lower temperatures typically produce nanotubes having thicker walls. Typical temperatures are between about 5° C. and about 75° C., such as between about 15° C. and about 50° C. The pH of the electrolyte solution is typically between about 0.1 to about 7, such as between about 3 and about 5.

In at least some implementations, the bath is agitated during all or a portion of the anodization process. Suitable means of agitation include magnetic or mechanical stirring. Ultrasonication can also be used to agitate the electrolyte solution.

Anodization is carried out for a sufficient time to form nanostructures having a desired length or other property, such as between about 1 minute and about 24 hours. Amorphous structures produced by such methods can be crystallized by annealing the nanostructures, such as by heating the nanotubes at a suitable temperature and a period of time of about 200° C. to about 1200° C. for about 10 minutes to about 7 hours.

According to another disclosed embodiment, nanotubes are prepared by treating a suitable metal oxide with alkali. For examples, titanium nanotubes may be prepared by treating titanium dioxide with about 13 wt % to about 65 wt % alkali, such as alkali or alkaline earth metal hydroxides, including sodium hydroxide and potassium hydroxide, at a temperature of between about 18° C. and about 160° C. In such examples, the diameter of the nanotubes is typically between about 5 nm and about 80 nm. The thickness of the nanotubes walls is typically between about 2 nm and about 10 nm, while the length of the nanotubes is typically between about 50 nm and about 150 nm. The titanium dioxide particles, in specific examples, have an average particle diameter of between about 2 nm and about 100 nm, such as between about 2 nm and about 30 nm. The properties of the resulting nanotubes, such as crystallinity and catalytic or electrical properties, can be modified by heating the nanotubes at about 200° C. to about 1200° C. for about 10 minutes to about 7 hours.

Another disclosed embodiment for forming nanotubes involves alkaline treatment of titanium oxide, titanium oxyhydroxide, or titanium hydroxide, followed by ionic exchange, such as by acid treatment, to obtain materials that include hydrogen titanates. The alkaline solution typically has a concentration of about 1 M to about 50 M, such as about 5 M to about 50M. Examples of alkaline materials which may be used to generate the solution include ammonium hydroxide, potassium hydroxide, sodium carbonate, and sodium hydroxide, as well as other alkali or alkaline earth metal hydroxides.

The alkaline titanium solution is heated, optionally with stirring, at about 50° C. to about 180° C., at a pressure of about 1 atm to about 150 atm, for about 1 hour to about 100 hours. The resulting product is treated with a dilute acid solution, such as about a 0.1 M to about 1M solution of an ammonium salt, such as ammonium carbonate or ammonium chloride, boric acid, chlorohydric acid, fluoric acid, nitric acid, phosphoric acid, or sulfuric acid until the pH of the solution is between about 1 and about 7, such as between about 2 and about 4. The solution is then held for about 1 hour to about 24 hours at a temperature of about 20° C. The resulting solid is then separated from this mixture, washed, and dried at temperature of about 60° C. to about 120° C. The structure of the material may be further adjusted by heating the material at a temperature between about 200° C. and about 500° C. in an atmosphere of one or more of oxygen, nitrogen, argon, hydrogen, and helium at a flow rate of about 0.11/min to about 1l/min.

Another disclosed embodiment for producing nanostructures involves forming a sol solution of a metal oxide or similar material, such as titanium oxide, in water, optionally with a co-solvent, such as a lower (e.g. 10 carbon atoms or fewer) alcohol, such as methanol or ethanol. The oxide is typically included in a concentration of about 2 wt % to about 50 wt %. The oxide particles typically have an average particle diameter of between about 2 nm and about 100 nm.

The resulting mixture is treated with a source of peroxide, such as hydrogen peroxide, to produce a peroxometal compound, such as peroxotitanic acid. In order to encourage all of the metal oxide to react with the peroxide, the peroxide typically is added in at least a 1:1 weight ratio to the metal oxide and is let stand for about 30 minutes to about 24 hours to dissolve the metal oxide. The mixture is then heated at about 50° C. to about 300° C., optionally with ammonium hydroxide, an organic base, or mixtures thereof.

An alkali metal hydroxide, such as LiOH, NaOH, KOH, RbOH, CsOH, or a mixture thereof, is added to the resulting solution in a ratio of alkali hydroxide to metal oxide of about 1:1 to about 30:1 or about 2:1 to about 25:1. This mixture is then heated at about 50° C. to about 350° C. The resulting material can be washed. In some examples, the wash is an acidic solution, such as a solution of a mineral acid, for example, hydrochloric acid or nitric acid. This material is then treated with a cation source at a ratio of cation source to metal oxide of about 1:1 to about 30:1 or about 2:1 to about 25:1 at a temperature of about 50° C. to about 350° C., such as about 80° C. to about 250° C. The cation source may be, for example, acids, non-alkali salts, and organic bases. Suitable acids include mineral acids, such as hydrochloric acid, nitric acid, and sulfuric acid. Organic acids may also be used, such as acetic acid, citric acid, glycolic acid, glycidic acid, maleic acid, malonic acid, and oxalic acid. Examples of organic bases usable in this technique include ammonium hydroxide; amines, such as monoethanolamine, diethanolamine, and triethanolamine; quaternary ammonium salts, such as tetramethylammonium salts; and derivatives or mixtures thereof. Examples of ammonium salts include ammonium acetate, ammonium chloride, ammonium nitrate, and ammonium sulfate. The cation-treated material is heated at about 50° C. to about 350° C., such as about 80° C. to about 250° C. The resulting material is then reduced in an inert gas, such as nitrogen, helium, neon, argon, krypton, xenon, or radon; along with a reducing gas, such as an amine, ammonia, hydrazine, or pyridine; or a hydrocarbon, such as methane, ethane, or propane, or mixtures thereof. The reduction is typically carried out at a temperature of about 100° C. to about 700° C.

Suitable nanostructures can also be produced by electrospinning, during which a core solution of an extractable or otherwise removable material, such as mineral oil, and a sheath solution of sheath material are subject to a high voltage. The solutions are thus charged and forced through a spinneret. In some embodiments, the sheath solution includes a viscosity modifying agent, such as a polymer. Suitable polymer materials include poly(vinyl pyrrolidone), polystyrene, polypropylene, polyethylene, polyvinyl chloride, polyvinylidene chloride, polyvinyl acetate, nylon polymers, polyurethane, and mono or poly functional (meth)acrylates. Additional polymer materials are disclosed in paragraph 51 of U.S. Patent Publication 2006/0223696, which is incorporated by reference herein.

Materials which can be used to form suitable sheaths include sol-gel precursors such as metal alkoxides; metal halides, such as metal chlorides, TiCl₄ for example; metal hydroxides; metal sulfates, such as Ti(SO₄)₂; metal acetylacetonates; and derivatives or combinations thereof. The metal alkoxides and metal acetylacetonates are typically tetrafunctional, the organic component being, in some examples, linear or branched C₁-C₁₂ alkoxides, such as methoxide, ethoxide, isopropoxide, n-butoxide, 2-butoxide, t-butoxide, n-hexoxide, 2-ethylhexoxide, 2-methoxy-1-ethoxide, acetylacetone, and mixtures thereof. In a particular example, the sheath material is Ti(OiPr)₄ (titanium tetraisopropoxide). Suitable solvents for these sols include lower alkyl alcohols (e.g. 10 carbon atoms or fewer), such as methanol, ethanol, isopropanol, n-butanol, cyclohexanol; solvents containing a carbonyl group, including aldehydes, such as acetone and cyclohexanone, ketones, such as methyl ethyl ketone, and esters, such as ethyl acetate; aromatic solvents, such as benzene, phenol, benzyl alcohol, and toluene; halogenated solvents, such as chloroform, methylene chloride, carbon tetrachloride, trichloroethane, hexafluoroisopropanol, and hexafluoroacetone; ethers, such as diethylether; other solvents, such as N-methylpyrrolidone, 1,3-dioxolan, acetonitrile, N-methyl-morpholine-N-oxide, tetrahydrofuran, N,N-dimethylformamide, pyridine, 1,4-dioxane, acetic acid, formic acid; and mixtures thereof.

In at least some embodiments, the core and sheath solutions are selected to be at least substantially immiscible. In yet further embodiments, the core and sheath solutions are at least somewhat miscible. The miscibility of the solvents can be used to adjust the porosity of the resultant nanostructures. For example, a core solution of polystyrene in N,N-dimethyl formamide and tetrahydrofuran can be used with an ethanolic solution of the structural material to produce highly porous nanostructures.

The core solution can be modified to include agents to coat the lumen of the sheath. For example, the core solution can include any of the sensitizing agents discussed in this disclosure. The properties of the nanotube surfaces can also be modified by adding appropriate agents to the core and sheath solutions. For example, alkylsilanes can be used to make a surface more hydrophobic.

Spinnerets suitable for use in forming nanostructures have a number of configurations, such as syringe-in-syringe arrangements; concentric spinnerets, such as using nested capillaries; or microchannels, such as branched microchannels. Suitable devices are disclosed in Srivasta, et al., “Electrospinning of hollow and core/sheath nanofibers using a microfluidic manifold,” Microfluid nanofluid DOI 10.1007/s10404-007-0177-0; Li et al., “Direct Fabrication of Composite and Ceramic Hollow Nanofibers by Electrospinning,” Nano Letters 5(4) 933-938 (2004); and U.S. Patent Publication 2006/0226580; each of which is incorporated by reference herein in its entirety.

The spinning conditions, including size and configuration of the spinnerets, the composition of the feed solutions, applied voltage, and solution feed rates can be adjusted to produce nanostructures having desired properties. For example, feed solutions having higher concentrations of structural materials can produce thicker structures, such as nanotubes having thicker sheaths. As a particular example, a feed rate of 0.03 mL/hour with a 1:10 weight ratio of metal to solvent can produce fibers having an average inner diameter of about 200 nm and a wall thickness of about 50 nm. The feed rates can also be used to vary the properties of the nanostructures. For example, higher flow rates of the feed solution can be used to produce structures having larger diameter lumens. In some examples, the feed rate of the structural solution is between about 0.1 ml/h and about 5 ml/h, such as between about 0.5 ml/h and about 1 ml/h or about 0.6 ml/h. Examples of feed rates for the core material are between about 0.01 ml/h and about 2 ml/h, such as about 0.05 ml/h to about 0.3 ml/h.

Suitable potentials, in some examples, are between about 2 kV and about 100 kV, such as between about 5 kV and about 30 kV or about 5 kV to about 20 kV. A suitable high-voltage power supply is the ES30P-5W, available from Gamma High Voltage Research, Inc., of Ormond Beach, Fla. The electrodes, in some examples, are placed between about 2 cm to about 30 cm from the outlets of the feed solutions, such as between about 5 cm and about 20 cm. The spinning process is typically carried out a temperature of between about 0° C. and about 50° C., such as between about 15° C. and 30° C. The temperature can be adjusted depending on the volatility and viscosity of solutions used in the spinning process.

Once the nanostructures are formed, the sheath material can be removed, such as by calcining the structures, such as at about 250° C. to about 800° C., such as about 500° C., for about 15 minutes to about 5 hours, such as for about one hour. The sheath material can also be extracted using a suitable solvent. Suitable solvents include non-polar organic solvents, such as alkanes, aromatic solvents, petroleum ether, or mixtures thereof. In a particular example, the core material is extracted using octane.

In yet another method, nanofibers are prepared by pressing a particulate form of the structural material, such as titania, at a pressure of between about 1 MPa and about 400 MPa and then sintering the resulting solid for about 6 hours at a temperature between about 1100° C. and about 1400° C. The sintered material is then heated in a mixture of inert gas and hydrogen (such as about 5% hydrogen and 95% inert gas, such as nitrogen) at a temperature of about 700° C. The flow rate of the gas is typically between about 100 ml/min and about 5000 ml/min or at least about 500 ml/min. Fibers formed by this method are typically between about 15-50 nm in diameter and about 5 μm long.

Nanowires can also be prepared by heating a mixture of structural material source, such as titanium halides, nitrates, alkoxides, or oxysulfates, with a polar organic solvent at about 170° C. to about 300° C. for between about 2 hours and about 24 hours, or at least about 5 hours. Suitable polar organic solvents include those having a dielectric constant of at least about 10 at 25° C. and a boiling point of at least about 100° C. One class of suitable solvents is alkylene glycols, including ethylene glycol.

Another method of forming nanowires involves coating a substrate, such as an acid etched substrate, such as a semiconductor, a metal foil, or a metal oxide, with a coating of nanocrystals of the desired structural material, such as nanocrystals of a metal oxide. The coated substrate is then treated with a solution including a metal salt, such as titanate, and at least one amine, phosphonic acid, carboxylic acid, or mixtures thereof. In a specific example, the solution includes methenamine. The solvent may be an organic or inorganic solvent, such as water. Suitable organic solvents include lower alkyl alcohols (e.g. 10 carbon atoms or fewer), such as ethanol. The solution is contacted with the substrate for a period of time sufficient to produce a desired length of nanostructure, such as about 30 minutes to about 6 hours. In some examples, the solution is heated at a temperature of about 60° C. to about 95° C.

Nanostructure Sensitization

According to some aspects of the present disclosure, the nanostructures have a sensitizing agent disposed on or within the nanostructure. In some embodiments, the sensitizing agent is applied to the nanostructure using one or more techniques such as chemical vapor deposition, evaporation, e-beam evaporation, electroless deposition (electroplating using chemical reduction of the sensitizing agent), electrodeposition (electroplating), ion plating, ion-assisted deposition, laser ablation, molecular beam epitaxy, screen printing, and sputtering. In a specific example, the sensitizing agent is applied using pulse electrolysis, such as pulse reverse electrolysis.

To electrodeposit the sensitizing agent according to an embodiment of the present disclosure, the sensitizing agent is added in the bath at a concentration of agent, such as metal ion, of about 0.05 mg/l to about 200 μl, such as between about 1 mg/l and about 25 μl or between about 10 mg/l and about 1 μl. The deposition bath can include other substances, including stress relievers, grain refiners, leveling agents, complexing agents, carriers, and brightening agents. Suitable electrolytic bath components include those described above for the preparation of unsensitized nanostructures by anodization, as described above.

In at least some implementations, the bath is agitated during all or a portion of the plating process. Suitable agitation processes include magnetic or mechanical stirring and/or ultrasonication.

The deposition process is typically carried out until a desired amount of material is deposited on the surface of the nanostructure. In at least some embodiments, the amount of time needed can be estimated by dividing the quantity of metal to be deposited by the current efficiency of the particular deposition process. In further embodiments, the amount of time needed to deposit a desired amount of sensitizing agent can be empirically determined.

The regularity of the deposition of the sensitizing agent can be affected by the throwing power of the bath solution. The throwing power can be adjusted as known in the art, such as by changing the pH of the bath or adding suitable agents.

In further embodiments, a nanostructure which includes the sensitizing agent is formed by anodizing the surface of a material, such as foil, containing the primary nanostructure material and the sensitizing material. A suitable voltage, such as between about 100 mV to about 40 V is applied to the anode for a period of time sufficient to produce a desired nanostructure, such as between about 1 minute and about 24 hours. Typically, longer periods of time are used to produce longer structures. The voltage and time period can be adjusted to produce nanostructures having different physical properties. For example, longer growth times at lower voltages can produce more stable, regular structures. In a particular example, when titanium nanotubes are used, the nanotubes are formed by anodizing a film of titanium and sensitizing agent for about 8 to about 10 minutes at about 10 V to produce nanotubes having a length of about 550 nm.

Amorphous nanostructures may be formed initially. Amorphous nanostructures may be converted to a crystalline state, such as by heating the nanostructures, such as in a high oxygen atmosphere, for a suitable time, such as between about 1 and about 24 hours, such as between about 4 hours and about 12 hours or between about 6 and about 10 hours. In some examples, the annealing is performed at a temperature of at least about 400° C., such as at least about 440° C., or about 450° C.

In order to improve the electrical properties of the resulting array, the nanostructures may be anodized, or otherwise treated, to form a passivated layer at the base of the nanostructures. For example, when the nanostructures are made of titania, they can be anodized in potassium hydroxide (such as about 0.05 M to about 1.0 M KOH) at about 26 V to about 76 V. This voltage can be stepped in increments, such as 3 V increments, with each step occurring when current density has stabilized after each step, such as at about 2 mA/cm². The inclusion of a passivated layer can help reduce electrical shorting of the array. This step may also be applied to nanostructures formed by other methods, such as nanostructures lacking a sensitizing agent.

Suitable electrolyte solutions include acidic electrolyte solutions, such as the combination of an acid and a fluoride, such as potassium fluoride or hydrogen fluoride. Suitable acids include acetic acid, chromic acid, phosphoric acid, sulfuric acid, disodium hydrogen phosphate, and dipotassium hydrogen phosphate. Basic electrolyte solutions can also be used, such as solutions of potassium hydroxide. The choice of electrolyte materials, and other bath components, can affect the properties of the nanostructures, such as their composition, shape, and stability. For example, fluoride ions can encourage tubular nanostructure growth. Acids, such as acetic acid, can aid in nanostructure stability.

In further methods, sensitizing agents are applied by chemically treating the nanostructures. For example, the nanostructure may contain exchangeable sites, such as weak Bronsted acid sites, such as surface metal-hydroxyl groups (M-OH). According to one embodiment of this method, a solution of the desired material, such as a salt of the sensitizing metal ion, is added to the nanostructure. The nanostructures are held in contact with the solution, optionally with agitation or sonication, for a period of time sufficient to exchange a desired number of the exchangeable sites. In some examples, nanostructures are contacted with the solution for between about 5 minutes and about 24 hours, such as between about 2 hours and about 6 hours. Additional substances, such as one or more complexing agents, may be added to the aqueous solution, such as to facilitate exchange. For example, ammonium hydroxide or another agent can be added to complex with the metal ions. In some examples, the solution of sensitizing agent is an aqueous solution. In further examples, the solution includes one or more organic solvents. In a particular example, an ethanolic solution of the sensitizing agent is used.

As previously mentioned, the nature, type of incorporation, and amount of sensitizing material can be altered to achieve different types of electrical properties and for particular applications. In some examples, the sensitizing agent is disposed on a surface of the nanostructure, such as coating the outer surface of the nanostructure or the lumen of a hollow nanostructure.

Electrode Formation

Electrodes are typically connected to the nanostructures after they have been formed on a substrate. Suitable electrode materials include aluminum, gold, indium-tin-oxide, fluorine-doped tin oxide, chromium, nickel, tungsten, palladium, platinum, ruthenium, or other metals, metal alloys, or mixtures thereof. In some examples, the electrode material is deposited on the nanostructure by sputtering or resistive thermal evaporation.

The electrode contacts can be connected to electrical devices, such as resistivity measurement apparatus, such as a potentiostat. Connections can be formed through any suitable means, such as silver paste/epoxy or wire bonding, such as ball bonding or wedge bonding.

Measurement Apparatus and Method of Use

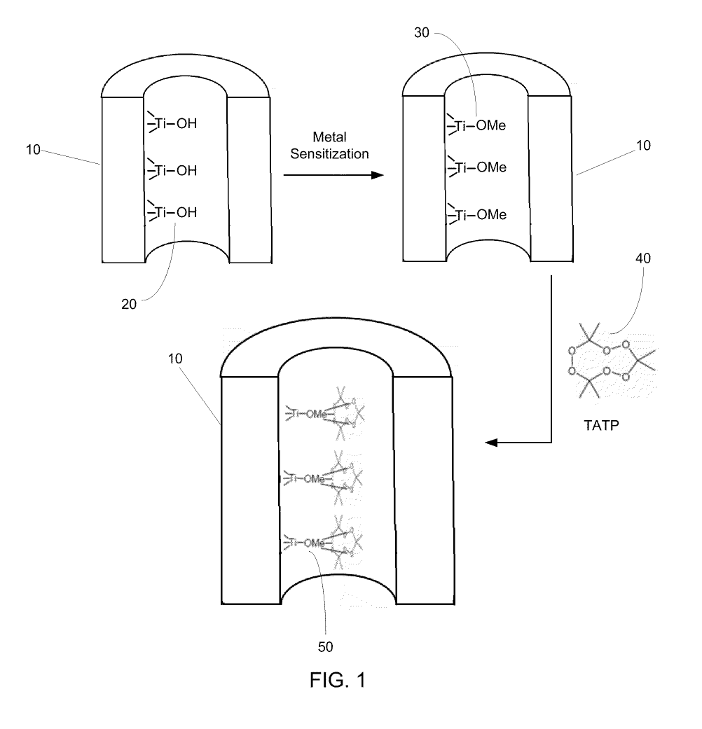

FIG. 1 is a schematic diagram illustrating how sensitized nanostructured arrays might be used to detect chemical substances, such as TATP. In the upper left panel, an unsensitized titania nanotube 10 is shown. The titania has exchangeable hydroxyl groups 20. At least a portion of these hydroxyl groups are exchanged with a sensitizing agent, such as a metal ion (represented in FIG. 1 as Me) 30, as shown in the upper right panel. The sensitized nanotubes are then exposed to a sample. In the bottom panel, a complex 50 of TATP molecules 40 and sensitizing metal ions 30 is formed. The interaction of TATP with the nanotubes alters the resistivity of the nanotube.

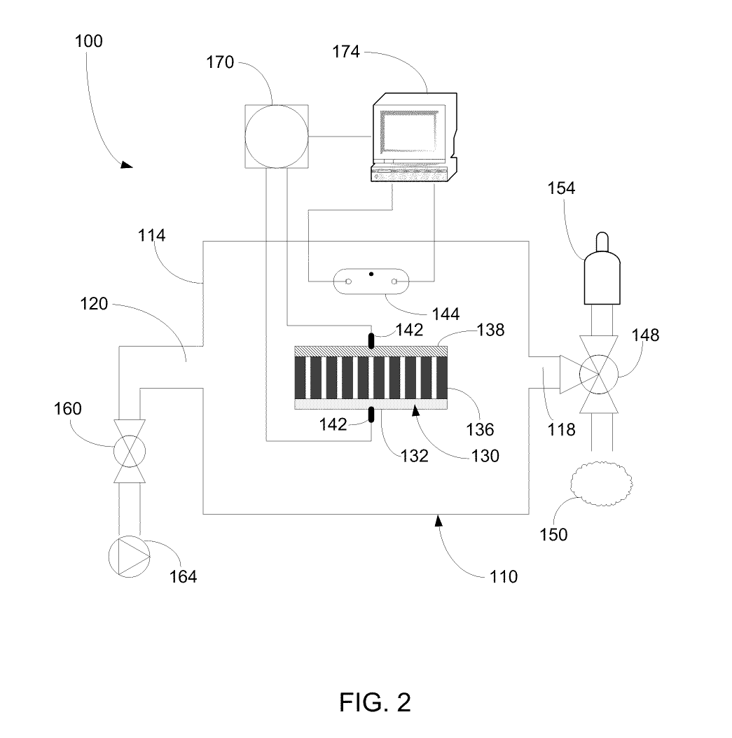

FIG. 2 illustrates an implementation of an apparatus according to the present disclosure useable to detect TATP. The apparatus 100 includes a test cell 110 having a housing 114. The housing 114 is generally formed from a material inert to TATP and other materials passed through the test cell 110. Suitable materials include plastics, metals, glasses, and ceramics. The test cell 110 further includes an inlet 118 and an outlet 120 through which samples and other materials may enter and exit the test cell 110, respectively. The test cell 110 may have other configurations, such as a single point of ingress/egress or a plurality of inlets 118 or outlets 120.

The test cell 110 includes a nanostructured array 130. The array 130 illustrated in FIG. 2 includes a metallic layer 132, such as a layer of titanium, nanostructures 136, such as titanium nanotubes, and a contacting layer 138, such as silver epoxy or a layer of gold or other conducting material. The metallic layer 132 and contacting layer 138 are electrically coupled to electrodes 142, such as platinum wires. In some examples, the electrodes are coupled to the array using silver epoxy. The array 130 may have other configurations, such as lacking the metallic layer 132 and having a plurality of contacting layers 138. Although the contacting layer 138 is shown as covering the entire surface of the nanostructures 136, the contacting layer 138 only covers a portion of the nanostructures 136 in further implementations.

The test cell 110 includes a lamp 144, such as a UV lamp. In further implementations, the lamp 144 is omitted. In yet further implementations, the test cell 110 includes a plurality of lamps 144.

The inlet 118 of the test cell 110 is in fluid communication with valve 148, such as a three way mixing valve. The valve 148 is also in communication with a sample source 150. The sample source 150 can be ambient air, a collected air sample, or a sample generated by other means, such as vaporization of a liquid sample or vapor generated from a solid sample. The sample source 150 may also be a standard or reference material. The valve 148 is further in communication with a carrier or purge gas source 154. Suitable purge gases include inert gasses such as nitrogen, helium, and argon. The valve 148 can be used to select whether the gas source 154, the sample source 150, or a mixture thereof is introduced into the test cell 110. In further configurations, the valve 148 or the gas source 154 are omitted.

The outlet 120 is in communication with a valve 160. The valve 160 is further in communication with a pump 164. In further implementations, one or more of the valve 160 and the pump 164 is omitted.

A controller 170, such as a potentiostat, is in electrical communication with the array 130 and may be used to send electrical signals to, and measure electrical signals received from, the array 130. A suitable controller is the PARSTAT 2273 potentiostat, available from AMETEK Princeton Applied Research, Inc., of Oakridge, Tenn. The controller 170 is in further communication with a workstation 174, such as a personal computer. In some examples, the controller 170 and workstation 174 are combined into a single component. In yet further embodiments, the workstation 174 is omitted.

The workstation 174 is also in communication with the lamp 144. In embodiments where the workstation 174 is omitted, the lamp 144 may be in communication with the controller 170 or another controlling device. Alternatively, the lamp 144 may be controlled through a manually operated input device, such as a toggle switch.

In operation, a sample is introduced into the sample source 150. Prior to such introduction, the test cell 110 may have been evacuated by the pump 164 or by passing purge gas through the test cell 110 from the purge gas source 154. The sample passes through the valve 148 and into the test cell 110 through the inlet 118.

A bias potential, such as a voltage of about 1 V to about 2 V, is applied between the metal surface 132 and the contacting layer 138 of the array 130. The resistivity of the array 130 is measured by the controller 170 as the sample passes through the test cell 110. The composition of the sample, such as the presence or concentration of TATP, can be determined from the measured resistivity, such as by comparing the measured response of the sample to a background measurement or a calibration curve prepared using samples of known concentrations.

In some examples, the sample is maintained in the test cell 110 until the measurement is complete. After completion, the sample can be purged by opening the valve 160 and either passing purge gas through the test cell 110 or pulling the sample from the test cell 110 by operation of the pump 164. In further implementations, the sample is continuously passed through the test cell 110, such as at a sufficient flow rate to allow detection of TATP in the sample as it moves through the test cell 110. Of course, these methods may be adjusted to take into account of differences in the configuration of the apparatus 100.

After a measurement is complete, the lamp 144 may be activated to clean the array 130. During cleaning, purge gas, air, or another material may be passed through the test cell 110 to help remove the prior sample from the array 130. The cleaning step is omitted in some methods.

### Example 1

### Synthesis of TiO₂ Nanotube-Arrays

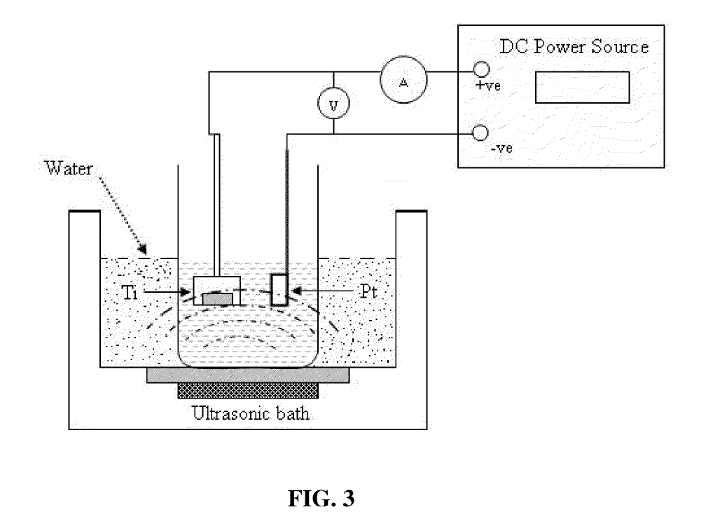

An electrolytic cell as show in FIG. 3 was used to generate a nanotube array. Titanium foil (0.2 mm thick, 99.9% purity, ESPI-metals, USA) was cleaned in acetone and dried under a nitrogen atmosphere. A small piece (2.0 cm²) of cleaned Ti foil was secured inside a polytetrafluoroethylene (PTFE) holder exposing only a 0.7 cm² area to a beaker containing 300 ml electrolytic solution. The electrolytic solution included 0.5 wt % ammonium fluoride (Fischer) in aqueous ethylene glycol (Fischer). The electrolytic cell was maintained in an ultrasonic bath (100 W, 42 KHz, Branson 2510R-MT). An external potential (20 V) was applied through a rectifier (Agilent E3640A). A flag shaped (e.g. rectangular) platinum electrode (thickness: 0.5 mm, area: 3.75 cm²) served as the cathode and the PTFE holder containing the Ti foil served as the anode. The distance between the two electrodes was kept constant (4-5 cm). The anodization current (1-3 mA), which can indicate successful formation of TiO₂ nanotubes, was monitored using a digital multimeter (METEX, MXD 4660A). The anodized sample was then cleaned to remove occluded solvents and electrolytes, such as by ultrasonicating the sample for 10 minutes in a mixture of water and ethanol (1:1 volume) followed by drying in a vacuum.

### Preparation of Metal Sensitized TiO₂ Nanotube-Arrays

Ion Exchange:

TiO₂ possesses weak Brönsted acid sites through the presence of surface hydroxyl groups (Ti—OH). These Brönsted acid sites can be selectively replaced by desired metal ions. In the ion exchange reaction, metal ion insertion is accomplished by mixing an aqueous solution of the ions in a mildly basic ammonia solution (pH 10 or above). The titania nanotubes were then introduced and stirred slowly for 12 hours at ambient temperature. The sensitized nanotubes were then cleaned and dried prior to further use.

Pulsed Electrodeposition:

The electrodeposition of different metal ions was conducted using pulsed electrodeposition. A Piro Tech APC-200 virtual instrument was used to record voltage and current during plating.

After sensitization, the TiO₂ nanotubes were annealed to form crystalline arrays.

### Use and Integration of the Detector Device

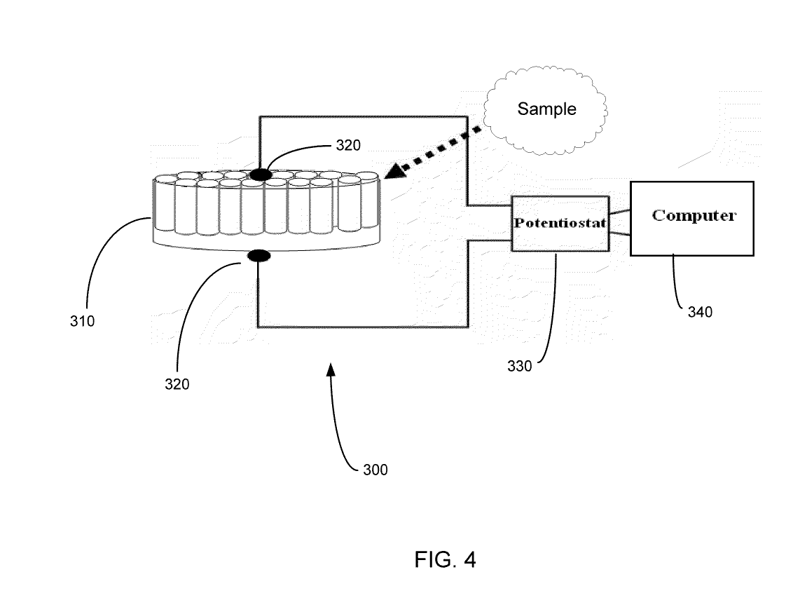

Silver epoxy was used connect to each face of the nanotubes array, as shown in FIG. 4. FIG. 4 illustrates a diction system 300 which includes array of nanotubes 310. The nanotubes 310 were connected to a circuit using silver epoxy 320. The silver-connected array served as a sensor. The metal ion sensitized TiO₂ nanotubular arrays were connected to a positive terminal and the Ti-foil was connected to the negative terminal of a controlled DC power supply, such as potentiostat 330. A suitable bias potential (in the order of 1-2 V) was applied between these two terminals. Data processing and display was accomplished using a computer 340.

The background resistance, R_{B} was measured under dry-air condition by recording the background current, I_{B}, at a bias potential V_{B} using Ohm's law, R_{B}=V_{B}/I_{B}. The sensor chamber was evacuated for a few hours before starting the measurements. The resistance was measured by passing dry-air through the sensor material and used as the baseline value. Dry-air containing a known concentration of TATP vapor served as the source for calibration. When peroxide explosive vapor was allowed to pass through the sensor material, it complexed with the metal ions (as shown in FIG. 1), and the change in resistivity of the nanotubes array measured. The resistance with different concentrations of TATP was used to optimize sensor set up and determine the sensitivity of the sensor. Measurements were carried out inside a glove box.

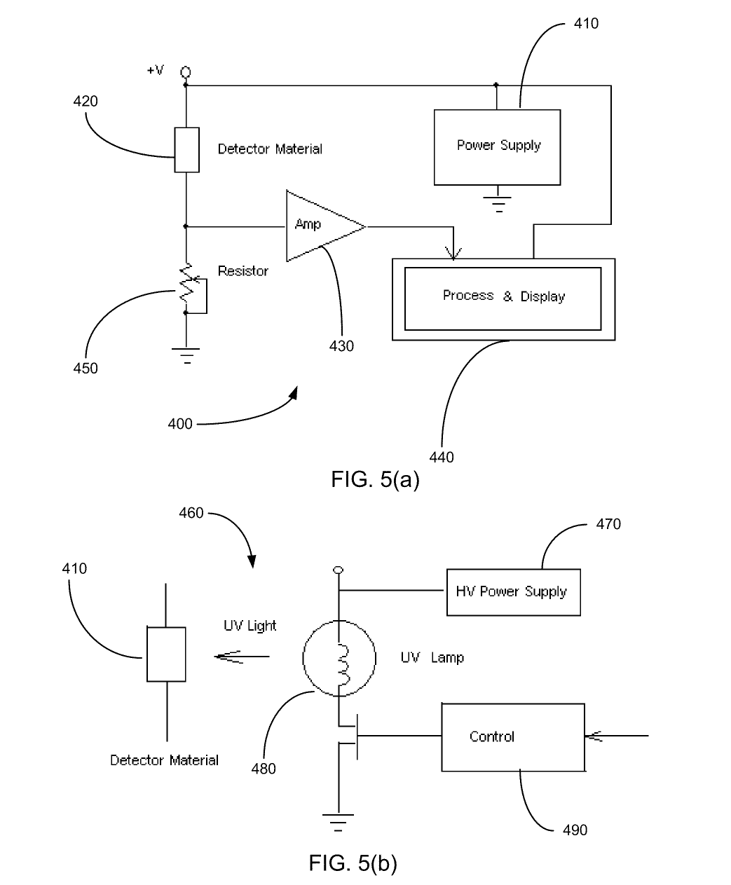

FIG. 5 illustrates circuit diagrams useable in the sensor of FIG. 4. FIG. 5(a) illustrates a detector circuit 400 useable with the sensor of FIG. 4. A power supply 410, such as a potentiostat, applied a voltage to a nanostructure array 420. The detected signal from the array 420 is passed to an amplifier 430. The amplified signal is received by a computer 440, which processes and displays the signal from array 420. The computer 440 also can be used to control the power supply 410. The circuit also include as resistor 450.

FIG. 5(b) illustrates a circuit 460 for operating a UV lamp that can be used to clean the array 410. A high voltage power supply 470 is electrically coupled to a UV lamp 480. The power supply 470 is controlled by a controller 490, which may be the computer 440 (FIG. 5(a)) or another controller, including manual controls such as toggle switches.

### Example 2

### Formation and Sensitization of Titanium Nanotubes Array

The growth of nanotubes was monitored as a function of increasing anodization time. The anodizing solution used for the experiments consisted of 0.5 M H₃PO₄ and 0.14 M NaF/NH₄F. Anodization was carried out at room temperature (22° C.-25° C.), with an anodization voltage of 20 V. Scanning Electron Microscopy (FESEM) images were obtained at various time intervals. One example is shown in FIG. 6, which illustrates a sample prepared by anodization for 45 minutes in fluoride solution.

In some specific implementations, the nanotubes array was covered by a fine layer of gold nanoparticles, which acts as electrical contacts for resistance measurements and helps provide uniform contact with electrodes. Gold was deposited using gold chloride solution. TiO₂ templates were immersed in the solution for 1-2 minutes. The sample was then dried under vacuum for 12 hours. The annealing was conducted in an argon atmosphere at 500° C. for 12 hours. Annealing decreases the number of defects inside the semiconductor titania, increasing the resistance of the array.

Resistance measurements were conducted using a setup similar to that shown in FIG. 4. The anodized titanium nanotube array was coated with silver epoxy on both upper and lower surfaces to provide electrical contacts. The silver epoxy was connected to platinum wires. The platinum wires, or other conducting wires, were then connected to the terminals of a potentiostat (Solartron SI1286, Farnborough, UK). The potentiostat was further connected to a display unit capable of showing current versus potential plots.

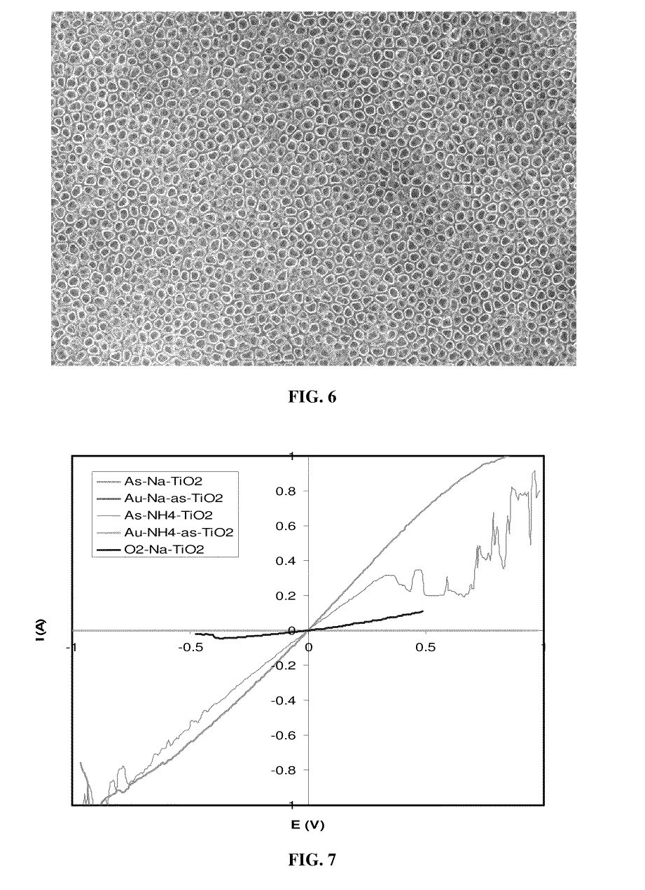

Current versus potential was measured for nanotube arrays formed under different conditions. The results are illustrated in FIG. 7. In FIG. 7, “as” refers to as-anodized arrays, “Na” refers to arrays anodized using sodium fluoride; “NH₄” refers to arrays anodized using ammonium fluoride; “Au” refers to gold coated arrays; and “O₂” refers to arrays annealed in the presence of oxygen. The uncoated arrays anodized in the presence of sodium fluoride or ammonium fluoride exhibited the least resistance; however, the response of the sodium fluoride array was more linear than that of the ammonium fluoride array. The gold-coated array did not conduct electricity. The array annealed in the presence of oxygen conducted electricity, but still had a very high resistance compared with the uncoated, unannealed arrays.

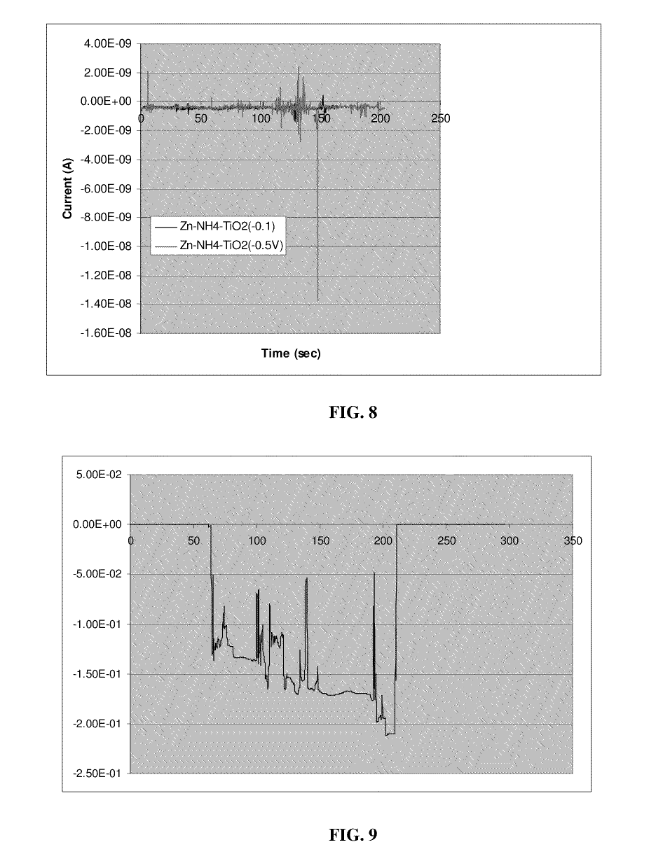

FIG. 8 is a plot of current versus time for a nanotubes array sensitized with Zn²⁻ ions in an Ar atmosphere (in a glove box). The resistivity remained fairly constant throughout the time the vapor was passed. Baseline resistivity in inert atmosphere was determined to be 10⁹ ohm-cm. The same sensitized array was then exposed to TATP vapor in a glove box under an argon atmosphere. As shown in FIG. 9, when TATP vapor was passed through the sensor under identical conditions, there was a sharp change in the resistivity to 10¹ ohm-cm. Thus, there was a 10⁸ order of magnitude change in current response in the TATP vapor compared to the baseline measurement.

### Example 3

### Synthesis of TiO₂ Nanotube-Arrays

An electrolytic cell as shown in FIG. 3 was used to generate a nanotube array. Titanium foil (0.2 mm thick, 99.9% purity, ESPI-metals, USA) was cleaned in acetone and dried under a nitrogen atmosphere. A small piece (2.0 cm²) of cleaned Ti foil was secured inside a polytetrafluoroethylene (PTFE) holder exposing only a 0.7 cm² area to a beaker containing 300 ml electrolytic solution. The electrolytic solution was prepared by mixing distilled water, 0.5 wt % ammonium fluoride (Fischer), and 0.5 M phosphoric acid. The pH was determined to be about 2.1.

A two-electrode configuration was used for anodization. The electrolytic cell was maintained in an ultrasonic bath (100 W, 42 KHz, Branson 2510R-MT). An external potential of 20 V was applied through a rectifier (Agilent E3640A). A flag shaped platinum electrode (thickness: 1 mm, area: 3.75 cm²) served as the cathode and the PTFE holder containing the Ti foil served as the anode. The distance between the two electrodes was maintained at 4.5 cm². The anodization current, which can indicate successful formation of TiO₂ nanotubes, was monitored using a digital multimeter (METEX, MXD 4660A). During anodization, ultrasonic waves were continuously irradiated into the solution. Anodization was carried out for 45 minutes.

The anodized sample was then cleaned to remove occluded solvents and electrolytes, such as by ultrasonicating the sample for 10 minutes in a mixture of water and ethanol (1:1 volume) followed by drying in an oven.

### Preparation of Metal Sensitized TiO₂ Nanotube-Arrays

Ion Exchange:

TiO₂ nanotube arrays were exchanged with Zn²⁺ using a 2×10⁻³ M ZnSO₄ solution. The titanium plate containing the titania nanotubes was suspended on the zinc solution and refluxed for 3 hours with constant stirring. The samples were then washed with de-ionized water three times, dried overnight under vacuum, and processed for characterization.

Characterization:

A field emission scanning electron microscope (FESEM; Hitachi, S-4700) was used to analyze the morphology of the nanotubes before and after functionalization. Energy dispersive X-ray spectroscopy (EDS) analysis was obtained using an Oxford detector. The TATP detection by the nanotubular detectors was carried out using the arrangement shown in FIG. 4. A standard silver epoxy connection (front and back, Chemtronics, USA) was given to the Zn²⁺—TiO₂ disc. The contacts were made by platinum wires which are hooked to the terminals of a potentiostat. The potentiostat was connected to a display unit which displayed the signal. Potentiostatic measurements were carried out using the CorrWare program (Scribner Associates, Southern Pines, N.C.) to determine whether TATP was present. TATP was introduced by placing TATP (5 mg) in a round bottom flask and allowing the vapors to pass through the detector through a glass nozzle (1 mm diameter).

Preparation of TATP:

50 mL of acetone was added to a 500 mL beaker. 30 mL of 30% hydrogen peroxide was added to the acetone with stirring. The beaker was placed in a salt-ice bath and cooled to 5° C. After cooling, 3 mL of 75% sulfuric acid was added dropwise to the beaker. The mixture was continuously stirred during the addition, with the temperature being kept between 5° C. and 10° C. The resulting mixture was kept in the ice bath for up to 24 hours. After sitting, a white crystalline precipitate was formed which was filtered and washed with 300-500 mL of water. The crystals were dried before further use.

### Results and Discussion

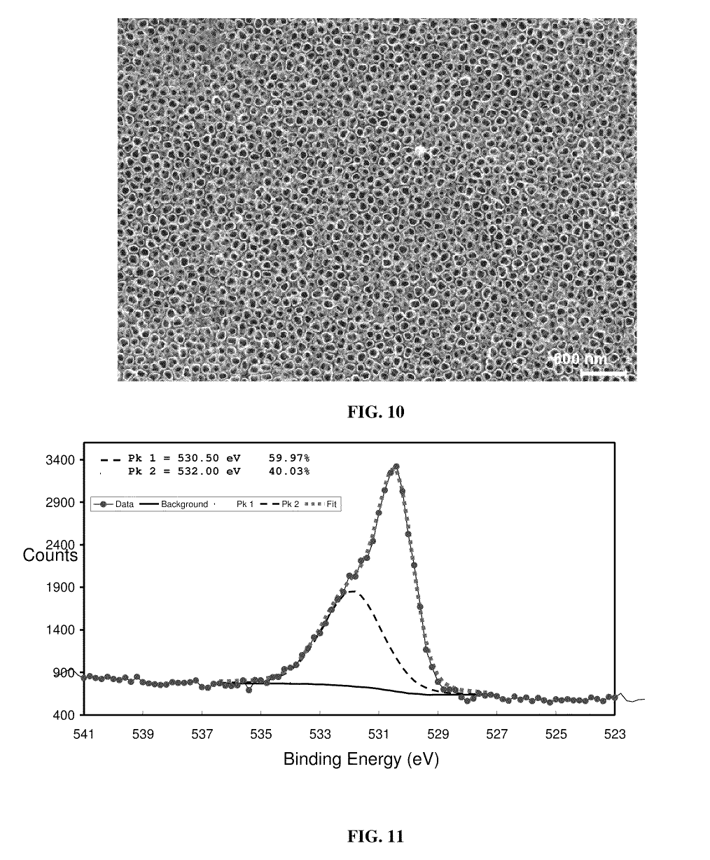

FIG. 10 shows the SEM images of zinc ion exchanged TiO₂ nanotubular arrays (Zn^{2\tilde{+}}TiO₂), which shows similar features to the parent TiO₂ nanotubular arrays. The average diameter of these nanotubes was found to be ˜80 nm, and the tube length ˜600 nm. The wall thickness of the titania nanotubes was found to be in the range of 15-20 nm. It was also observed from FIG. 10 that TiO₂ nanotubes were compact (nanotubes were well attached to each other) and one dimensionally oriented (straight). There was no surface deposition of zinc salt observed on the sample. The structural integrity and morphology of the titania nanotubes also remained undamaged after the ion exchange process. EDX (energy dispersive X-ray) analysis showed the presence of Zn²⁺ ion (0.65 wt %) in the sample.

The presence of large numbers of titanyl (Ti—OH) groups at the surface of the nanotubes was confirmed by XPS analysis. FIG. 11 shows the O1s XPS spectrum of the as-anodized titania nanotubes. The spectrum exhibits a major peak at 530.5 eV and a shoulder at 532.0 eV corresponding to TiO₂ and TĩOH. The spectrum shows that almost 40% of the surface was covered by titanyl groups, which are Brönsted acidic in nature and are known to be exchangeable sites. The presence of an increased number of these acidic sites in titania nanotubes compared with nanoparticles (14-16%) may be due to the high surface area and greater roughness (onion ring structure) in the former compared to the latter. The number of sites reduces to 10-15% after annealing the nanotubes, likely due to the condensation of the titanyl groups. Even though both annealed and as-anodized titania nanotubes can be used to exchange the metal ions, the latter is preferred for this purpose due the presence of high density titanyl groups on the surface. The exchange process of the surface titanyl proton with zinc ions is shown in equation below:

2TĩOH+Zn²⁺=2TĩOZn+2H⁺

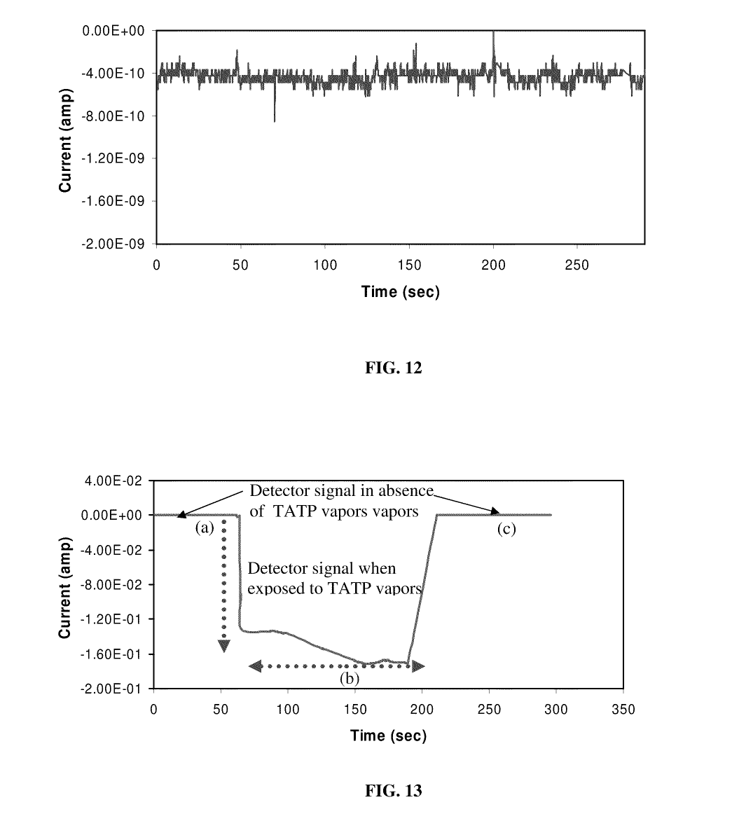

Tests of the sensing properties of the Zn⁺²TiO₂ material were carried out inside a glove box under argon (Ar) atmosphere (experimental set up shown in FIG. 4). The potentiostatic measurements were carried out at 0.5 V for Zn^{2\tilde{+}}TiO₂ with and without passing TATP vapors. Base line current (amp) was measured without passing TATP vapors through the sensor material and was found to be in the order of 10^{˜8}-10^{˜9} amp, which corresponded to resistivity of 10⁸ to 10⁹ ohm-cm (FIG. 12). These results indicated that there was no change in current with time in the absence of TATP (i.e. a smooth base line is obtained of the detector material at −0.5V). Although this test was carried out at −0.5V, other potentials in the range of 3.5V to −3.5 V also can be used for this measurement.

When TATP vapor was passed through the material under identical conditions, such as in a glove box under an Ar atmosphere for 300 seconds, there was a sharp change in the current to 10^{˜1} to 10^{˜3} amp (corresponding to a resistivity of 10¹ to 10³ ohm-cm (FIG. 13). The actual signal was the current (I) obtained by applying a constant potential (V, 0.5 V for this test) plotted vs. time (t). FIG. 13 shows three phases: phase (a) shows the current obtained before passing TATP vapors; phase (b) shows the current obtained when the material is exposed to TATP; and phase (c) shows the current obtained after the removal of TATP from the material. The increase in negative current shows the adsorption of TATP into the titania nanotubes sensor. Removal of the TATP source brought the current back to its original value.

The current remained higher throughout the time when the TATP vapors were passed through the sample. The fluctuation in the current density obtained when TATP vapor passed through the material is due to the solid source used as the vapor precursor. The vapor released from the solid source is not constant over time. However, even with the fluctuation, the measured signal was well beyond the number obtained from the detector material when no TATP vapor was passed through the material.

Without intending to be bound by theory, it appears that the increase in current may be due to the coordination of the oxygen sites of the TATP molecule with the zinc ions (FIG. 1). The lone pair electrons from the oxygen sites are released to the vacant d-orbitals of the zinc ions and through to the nanotubes. This increase in electron pool in the material helps increase the current of the overall sensor material (further aided by the interconnecting titania nanotubes), which is signaled through an increase in negative current in the display. As the measurements were carried out inside the glove box with controlled atmosphere, the interference of other oxygen ligated compounds are omitted and the signal change is due to the TATP molecules only. The material appears to be highly sensitive, as only 5-10 mg of TATP was used for this measurement. The vapor pressure generated from this small amount of TATP was expected to be very low, but was still detected by the prepared material with a good signal obtained.

These wafer sensors are auto regenerated, as confirmed from FIG. 13. The original current of the base sensor material reappears when TATP vapor was no longer present. This auto regeneration may be due the decomposition of the TATP molecules on the titania surface. Although this Example demonstrates the activity of zinc ions in detecting TATP, other cations, such as Li¹⁺, Na¹⁺, Cd²⁺, etc., also can be useful for this purpose.

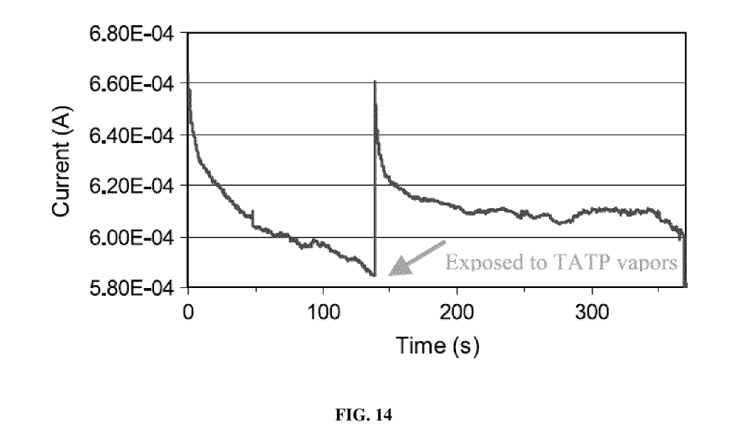

A test similar to that described above was performed except under ambient conditions, in the presence of air, rather than in an argon atmosphere. A similar change in signal was observed similar to the argon atmosphere test when the sensitized nanotubes were exposed to TATP vapors under ambient conditions (FIG. 14). The applied potential was limited to 0.05 V due to the high current response under ambient conditions. The signal strength was lower in magnitude compared to the test carried out under controlled atmosphere.

It is to be understood that the above discussion provides a detailed description of various embodiments. The above descriptions will enable those skilled in the art to make many departures from the particular examples described above to provide apparatuses constructed in accordance with the present disclosure. The embodiments are illustrative, and not intended to limit the scope of the present disclosure. The scope of the present disclosure is rather to be determined by the scope of the claims as issued and equivalents thereto.

What is claimed is:

1. A method of detecting triacetone triperoxide, comprising:
providing an array of titanium nanostructures comprising a sensitizing agent;
contacting the array with a fluid sample; and
measuring the resistance of the array.

2. The method of claim 1, wherein the nanostructures comprise titania nanotubes.

3. The method of claim 1, wherein the sensitizing agent is selected from Li⁺, Cu²⁺, In³⁺, Sb³⁺, Sc³⁺, NH₄⁺, Na⁺, Zn²⁺, Cd²⁺, and Ti⁴⁺.

4. The method of claim 1, where the nanostructures include a sensitizing agent coating on at least a portion of a surface thereof.

5. The method of claim 1, wherein the sensitizing agent is incorporated into the nanostructures.

6. The method of claim 1, carried out in an inert atmosphere.

7. The method of claim 1, further comprising irradiating the array with ultraviolet radiation.

8. A triacetone triperoxide sensor comprising:
an array of nanostructures, at least a portion of the nanostructures comprising a sensitizer selected from the group consisting of Li⁺, Cu²⁺, In³⁺, Sb³⁺, Sc³⁺, NH₄⁺, Na⁺, Zn²⁺, Cd²⁺, and Ti⁴⁺; and
a plurality of electrical contacts electrically coupled to the array, the electrical contacts configured to apply a voltage to the array and measure an electrical property of the array.

9. The sensor of claim 8, wherein the sensitizer is coated on a surface of the nanostructures.

10. The sensor of claim 8, wherein the nanostructures are hollow.

11. The sensor of claim 10, wherein the sensitizer is disposed on a luminal surface of the nanostructures.

12. The sensor of claim 8, wherein the sensitizer is incorporated into the nanostructures.

13. The sensor of claim 8, further comprising an ultraviolet lamp.

14. An apparatus for detecting triacetone triperoxide, the apparatus comprising:
a housing, the housing comprising an inlet;
a sensor comprising the sensor of claim 8 disposed in the housing; and
a controller in electrical communication with the sensor.

15. The apparatus of claim 14, further comprising an ultraviolet lamp disposed in the housing.

16. The apparatus of claim 14, wherein the controller comprises a potentiostat and the housing comprises an outlet, the apparatus further comprising:
an ultraviolet lamp disclosed in the housing;
a pump in communication with the outlet; and
a purge gas source in communication with the inlet.

17. A method of detecting a chemical substance comprising:
providing an array of nanostructures, the nanostructures comprising a peroxide-complexing sensitizing agent;
contacting the array with a fluid sample; and
measuring the resistance of the array.

18. The method of claim 17, wherein the nanostructures comprise TiO₂.

19. The method of claim 17, wherein the nanostructures comprise TiO₂ nanotubes.

20. The method of claim 17, wherein the peroxide-complexing sensitizing agent is selected from the group consisting of Li⁺, Cu²⁺, In³⁺, Sb³⁺, Sc³⁺, NH₄⁺, Na⁺, Zn²⁺, Cd²⁺, and Ti⁴⁺.

21. The method of claim of claim 17 further comprising treating an unsensitized array of nanostructures to produce the array of nanostructures by exchanging at least a portion of exchangeable sites on the unsensitized array with the peroxide-complexing sensitizing agent.
